(19) United States
(12) Patent Application Publication
(10) Pub. No.: US 2004/0054255 A1
(43) Pub. Date: Mar. 18, 2004

(54) Endoscopic instrument for use in cavities

(76) Inventors: Christian Pilgrim, Goch (DE); Jurgen Sterzel, Siegen (DE); Matthias Hillebrand, Kreuztal (DE)

Correspondence Address: CANTOR COLBURN, LLP, 55 GRIFFIN ROAD SOUTH, BLOOMFIELD, CT, 06002

(21) Appl. No.: 10/399,925

(22) PCT Filed: Oct. 1, 2001
(86) PCT No.: PCT/EP01/11332

(30) Foreign Application Priority Data
Oct. 24, 2000 (DE) 100 52 863.5

(51) Int. Cl. A61B 1/04

(52) U.S. Cl. 600/109

(57) ABSTRACT

An apparatus for viewing an object includes a solid-state colour filter (40) and an imager (18) such as a charge coupled device. The solid state colour filter (40) is operable to produce portions of an optical image of the object (20). The imager (18) is operable to receive the portions of the image and to respond by generating sequential sets of electrical signals, wherein each set of electrical signals respresents one of the differently coloured, single colour portions of the optical image.

The present invention concerns an endoscopic instrument for use in hollow spaces with at least one sensor unit element for accommodating optical systems having image information.

Numerous configurations of endoscopes are known in the state of the art. In the framework of diagnostic and/or therapeutic methods (endoscopy), body cavities and canals are directly observed with the aid of endoscopes. In the area of technical applications, endoscopic instruments are used, for example, for the observation and analysis of cracks and the like in vane wheels and atomizing chambers of turbines.

Usually endoscopes include an optical system of prisms and/or lenses, as well as an illumination system, which are often arranged in rigid tubes or flexible hoses as a function of use. Thus, for example, the optical system with so-called fibroscopes (called fiber endoscope) contain an image guidance system arranged in a flexible tube in the form of a pliable fiber optics of glass fiber bundles through which the image to be recorded is transmitted. Furthermore, endoscopic instruments are known whose optical system has sensor unit elements for the electronic registration of images. The observation of hollow spaces succeeds through direct observation of optical and/or electronic processing of the image recorded using the optical system.

With so-called video-endoscopes, the image-recording sensor element is introduced into the hollow space. In contrast, with fibroscopes, the image to be recorded is transmitted through fiber optics and is directly observed outside the hollow space or gathered by a sensor element and usually brought to the indicator electronically edited.

As a rule, endoscopic instruments use so-called CCDs (Charge-Coupled Devices), charge-coupled components or circuits for the electronic gathering, editing and preprocessing of images in hollow spaces that are arranged matrix-like for a surface-wise image digitizing. By using mosaic color filters with which the matrices can be provided, color images are receivable. With the customarily used sensor element for registering images, color value signal-generating mosaic color filters such as RGB (red, green, blue) or CMYK (cyan, magenta, yellow, black) are used for generating color images.

At the present time, high resolution color CCDs for use in endoscopic instruments are known in the state of the art that can be incorporated into the distal ends of flexible endoscopes with 90° prisms/lens systems, among other things. A 1/10 inch large CCD with 270,000 pixels (image dots) or a ⅙ inch large CCD with 410,000 pixels (image dots) are used in endoscopes with an outside diameter from 6 mm to 13 mm. The latter is damped with a mosaic color filter and outfitted with 410,000 microlenses for straight line signal conductance. Moreover, at this time, a CCD with 850,000 pixels damped with an RGB filter is available which measures ⅓ of an inch in the diagonals and therefore can only be used in endoscopes with an outside diameter of ca. 10 mm or greater.

Using CMOS (Complementary Metal Oxide Semiconductor) components instead of CCDs is known from WO 99/58044. Nonetheless, CMOS components have similar disadvantages to CCDs for use in endoscopic instruments since the spectral and local resolution is restricted by the use of mosaic filters like those of CCDs.

The demands imposed upon endoscopic instruments for investigating hollow spaces increase more and more. Thus, endoscopic instruments in medicine and technology must not only occasionally make multidimensional representations, but must also be able to conduct minimally invasive operations and analyses in hollow spaces, especially with regard to different optical properties, as well as simultaneously observing process sequences, especially without image-falsifying image decomposition and image overlays for these observations. In order to do justice to these demands, there exists a continuing need for economical endoscopic instruments that enable a qualitatively high grade image gathering with high spectral and local resolution, high sensitivity and high brightness dynamics with the smallest possible dimensions, especially with regard to the outside diameter.

In view of this state of the art, the invention is based on the objective of furnishing an endoscopic instrument for use in hollow spaces of the type mentioned at the beginning that can be manufactured economically and simply while increasing the recordable spectral range and local resolution, sensitivity and brightness dynamics.

The objective is accomplished in accordance with the invention in that the sensor element consists of an arrangement of image dot units out of which the image information acting on the sensor element in the form of electromagnetic radiation can be assembled, whereby the image dot units are structured axially in relation to the direction of incidence of the electromagnetic radiation onto the sensor element.

Advantageously, at least two pieces of image information are detectable in an image dot unit for each image dot. In a concrete configuration, the sensor element has at least two layers, a basically area-covering sensor layer and a layer serving for signal preprocessing and processing adjoining thereto in the direction of incidence of the electromagnetic radiation.

The use in accordance with the invention of image dot units structured axially in relation to the direction of incidence of the electromagnetic radiation striking upon the sensor element, thus sensor units with image dot units that are structured horizontally or vertically in their layer construction according to the position of the sensor element, makes possible furnishing endoscopic instruments for use in hollow spaces that have a greater resolution and sensitivity with smaller dimensions than the previously known endoscopic instruments. The axially structured image dot units of the invention make it possible (in contrast to the sensor elements previously used in endoscopic instruments, for example, with CCDs acted upon by mosaic filters) to use the entire surface of the sensor element for gathering image information. Advantageously, the ratio of the surface provided for image information gathering to the overall surface of the sensor element lies in a range from 0.8 to 1 and more preferably amounts to 1.

Advantageously, the axially structured image dots are sensitive in at least two different spectral ranges, that is, sensitive within different spectral ranges. In this connection, one also speaks of polychrome image dot units. These polychrome image units are consequently structured horizontally or vertically in their layer construction. Horizontally or vertically structured image dot units are, moreover, utilized in the endoscopic unit according to the position used. The position of the horizontally or vertically structured image dot unites is always selected so that the structuring is axial in relation to the direction of incidence of the electromagnetic radiation onto the surface of the sensor element.

Image dot units or pixels horizontally structured in their layer construction or their layer sequences are known, for example, from WO 99/00848, to the disclosure of which reference is made herewith. Advantageously, sensor elements in accordance with WO 99/00848 are used in the endoscopic instrument of the invention, thus sensor elements for electromagnetic radiation formed by a structure of an integrated circuit, especially an ASIC (Application Specific Integrated Circuit) on the surface of which a layer sequence sensitive to electromagnetic radiation is installed consisting of an arrangement of image dot units, whereby each image dot unit has a radiation transducer in the form of the layer sequence mentioned for transforming the incident radiation into an intensity-dependent measured value, and resources for gathering and storing the measured value, and whereby a control facility is provided for reading out the measured values related in any given case to an image dot unit so that the image beamed onto the sensor can be composed from the image dot unit-related measured values. Such a sensor element is usually constructed in so-called TFA technology (Thin Film on ASIC), as is known, for example, from the article “Thin Film on ASIC [:] A Novel Concept for Intelligent Image Sensors,” by H. Fischer, J. Schulte, J. Giehl, M. Böhm and J. P. M. Schmidt from the year 1992 (cf. Mat. Res. Soc. Symp. Proc. Vol. 285, p. 1139ff). By using polchrome horizontally structured (PHS) image dot units or pixels, especially when using TFA technology, there exists the possibility of detecting several color channels in a single horizontally layered image dot unit (pixel). The image resolution of spectrally restricting mosaic color filters is therewith superfluous.

Furthermore, image dot units of the invention vertically structured in their layer construction or in their layer sequenced can be used as a sensor element in an endoscopic instrument as the latter are known, for example, from the article “Three dimensional metallization for vertically integrated circuits” by P. Ramm et al. in Microelectronic Engineering 37/38 from the year 1997 (cf. pp. 39-47). In an advantageous configuration of the invention, corresponding polychrome vertically structured image dot units are used.

To increase the spectral sensitivity of the sensor element, especially in the near infrared region (NIR region) and for improving the transient properties of the sensor element, at least one further opto-electronic transducer sensitive toward electromagnetic radiation is allocated to each image dot unit in accordance with an advantageous refinement of the invention, which is a component of the integrated circuit. Advantageously, the opto-electronic transducer is a component constructed preferably of crystalline silicon or other suitable semiconductor materials (cf. WO 00/52759), for example, a photo-diode, a photo-gate, photo-transistor or the like. Opto-electronic transducers of this sort are known, for example, from WO 99/00848, to the disclosure of which reference is made. In a further advantageous refinement of the invention, the sensor element of the endoscopic instrument for use in hollow spaces has a spectrally controllable sensitivity for which multiple layer systems of pilin, pipilin or similar layer form types are preferably used, as are known from DE 44 41 444, DE 196 37 126 and DE 197 10 134, to the disclosure of which reference is made. In a particularly advantageous refinement of the invention, the sensor elements are sensitive in the ultraviolet range. In this way, the contrast in connection with images to be recorded is increased, in particular, even with unprocessed or uncolored objects or preparations to be examined.

In an especially advantageous refinement of the invention, the ASIC includes an apparatus for image editing and/or evaluation as a component of the integrated circuit, preferably an apparatus for noise suppression, preferably by image summation and/or averaging, amplifiers, preferably so-called lock-in amplifiers, or the like. Lock-in amplifiers are known in the state of the art and are used to detect an interesting signal with a specific frequency and phase from a noisy signal. In spectroscopy, lock-in amplifiers are typically used for amplification and editing of small optical signals in order to detect weaker optical signals against a background noise. The endoscopic instrument of the invention advantageously has a lock-in amplifier for amplification and editing of detected image formations. Advantageously, a lock-in amplifier is moreover provided for each image dot unit and is a component of the integrated circuit.

Advantageously, a signal processing is arranged on the sensor element beside the integrated circuit (image preprocessing). In this way, signal preprocessing and/or processing becomes more rapid and economical since otherwise external computing facilities or processors used for signal processing and/or preprocessing can be dispensed with or need merely be designed for a preprocessing and/or processing of narrow band signals, since the signals are already preprocessed and/or processed on chip. Signal preprocessing and/or processing in this way becomes less susceptible to disturbance. A signal transmission from the sensor element to the signal preprocessing facility on chip basically has a shorter communications pathway. In this way, connections or interfaces for connection with an external signal processing facility in particular can be dispensed with, which otherwise would enlarge the dimensions of the endoscopic instrument.

In a further especially advantageous refinement of the invention, the sensor element of the endoscopic instrument for use in hollow spaces has a radiation-emitting structure that serves for irradiating the hollow space to be examined or analyzed. Advantageously, the radiation-emitting structure is constructed as an illumination facility for illuminating the hollow spaces to be examined. In a preferred refinement, the radiation-emitting structure is one or several directly or indirectly emitting diodes, preferably, a diode emitting radiation of various wave lengths. Advantageously, the sensor elements and the radiation-emitting structure are adapted to one another with respect to their spectral ranges, preferably such that the sensor elements are spectrally sensitive in the range of the radiation emitted by the radiation-emitting structures and/or radiation called forth by the emitted radiation, for example, for luminescence phenomena, such as with fluorescence or phosphorescence.

In a further configuration of the invention, the radiation-emitting structure makes possible a sequential irradiation of the hollow space to be examined, or a part of it. Furthermore, radiation of various wave lengths is advantageously beamed in a specified sequence, for example, in a red, green, blue sequence, whose interaction products are detected or read out with the object to be observed as image information by the individually structured image dot units of the sensor elements. The reading out or detection takes place, moreover, serially with respect to the color information on the individual image dot, parallel with respect to locality and intensity information of the image dots detecting a selfsame color, and therewith very rapidly. Furthermore, mosaic filters previously used in the state of the art can thus be dispensed with in accordance with the invention.

In a further especially advantageous refinement of the invention, white light, as well as luminescence observations, can be actualized with an endoscopic instrument. In addition, especially simple and sensitive fluorescence video endoscopes can be constructed using polychrome horizontal or vertical structures. The use of structures emitting various radiation makes possible selective examination of further individual regions of the hollow space to be examined, for example, by marking or recognition of [intelligible] areas by selective irradiation with radiation of different wave lengths, for example, for recognition of specific tissue changes on the basis of different fluorescence behavior.

The use of position reporters on the distal end of the endoscopic instrument of the invention advantageously allows an exact location in the frame of multimodal matching methods or the like, in which, for example, endoscopic and/or nuclear spin topographic volume data sets are brought into exact fitting superposition. In this way, multiple dimensional diagnosis vectors can be determined that, in particular, permit improved diagnosis and treatment. In addition, multiple dimensional planning and implementation in connection with simultaneous spatial control using the endoscopic instrument advantageously exists when using an endoscopic instrument of the invention so that risks are minimized during the operation and shorter operation times can also be realized, and consequently, overall a reduction in operation costs can be realized.

In a further advantageous refinement of the invention, the endoscopic instrument is constructed as a capsular probe that is moved with a preferably integrated drive actively and/or passively through peristaltic movements of organs of the body to be examined, especially wavelike progressive contraction of the intestine or the esophagus, independently or remotely controllable in and/or through hollow spaces. For this, the endoscopic instrument advantageously has a computing unit on the integrated circuit for control and/or remote control of a drive, as well as a facility for non-contact transmission of recorded image information to a separate image information receiving facility situated outside the hollow space, on the part of which the image information is indicated to the user of the endoscopic instrument. Transponder technology or the like can be used for this, for example. In a further advantageous refinement of the invention, the integrated circuit of the endoscopic instrument is read out to the user on the part of a correspondingly suitable indicator facility, for example, a monitor or the like for displaying the image information after passing through a hollow space to be examined and removal of the endoscopic instrument from the hollow space. In an especially advantageous configuration of the invention, the endoscopic instrument constructed as a probe is constructed as a component completely by means of semiconductor engineering production methods.

Advantageously layer systems are used in the endoscopic instrument for the sensor elements that are tempered during the production process following primary manufacture. This treatment leads to a healing of bonding fissures in the structure of the layer system of the sensor elements of amorphous silicon. The endoscopic instrument of the invention is thus autoclavable, which is in particular necessary with medical uses, but which has not been possible with previously used CCD sensor elements and the morphological changes in these components resulting from this under the action of temperature.

In comparison with the state of the art, the endoscopic instrument of the present invention has numerous advantages, as will be explained below by way of examples.

Owing to the possibility of conducting a signal processing on the part of the integrated circuit of the endoscopic instrument (image preprocessing), an at least semiautomatic evaluation of examination images with subsequent selective double checking by the user is possible. In this way, shorter examination times, a lower stress on the patient as well as an improved diagnosis result combined with reduced repeat examinations and the presence of a lower probability of overlooked findings.

Due to the smaller construction dimensions of the endoscopic instrument of the invention, endoscopic examinations of narrow passage systems, such as bile ducts and like structures are possible.

The combination of the invention of endoscopic instruments and radiation-emitting structures with various wave lengths, especially for the observation of luminescence phenomena, such as fluorescence and/or phosphorescence, permit an analysis of tissue changes, and therewith an improved treatment and/or therapy in a simple and economical manner.

By combination with additional sensor elements, position reporting facilities and the like on the distal end of the endoscopic instrument, a partially automated high precision spatial scanning of the hollow spaces to be examined is available.

Through the use according to the invention of sensor facilities, preferably on the distal end of the endoscopic instrument, different measured values, in particular biological factors, can be ascertained and evaluated parallel to one another in one examination process. In this way, a combination diagnosis with high specificity or sensitivity and reliability exists.

Owing to the improved detail resolution of the endoscopic instrument, tissue changes and the like, especially carcinomata, can be established more easily and earlier. Through an earlier treatment possibility associated with this, healing chances are greater and treatment costs are lower.

Due to the smaller dimensions of the sensor element of the endoscopic instrument of the invention, there remains space for enlarged instrumentation channels, for example, for the use of tools for mechanical access in the framework of therapeutic interventions, with identical resolution and identical outside dimensions on the part of the endoscopic instrument.

Further details, features and advantages of the invention will be explained in greater detail below on the basis of the designs represented in the Figures, wherein:

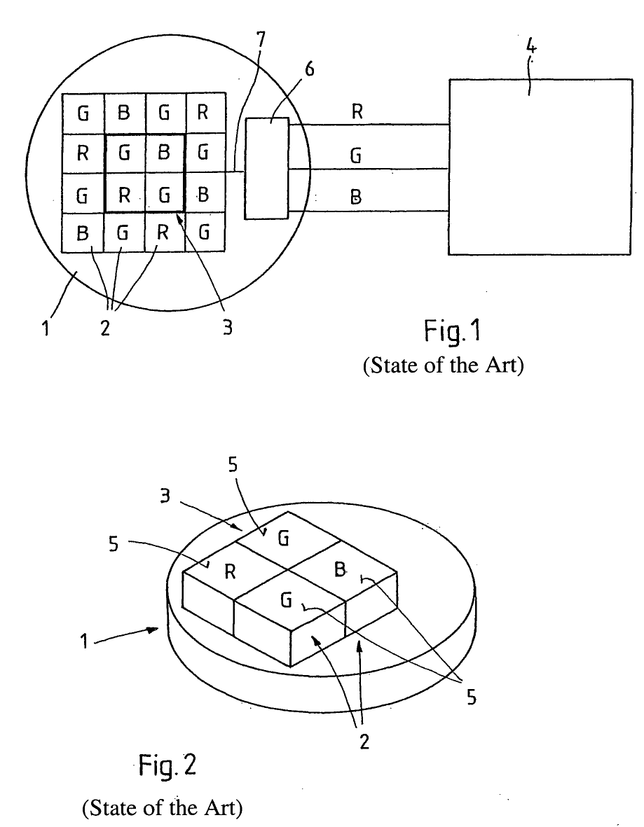

FIG. 1 Illustrates the principal structure of a CCD used as a sensor element in endoscopic instruments in accordance with the state of the art;

FIG. 2 Illustrates in a schematic perspective view the sensor element according to FIG. 1;

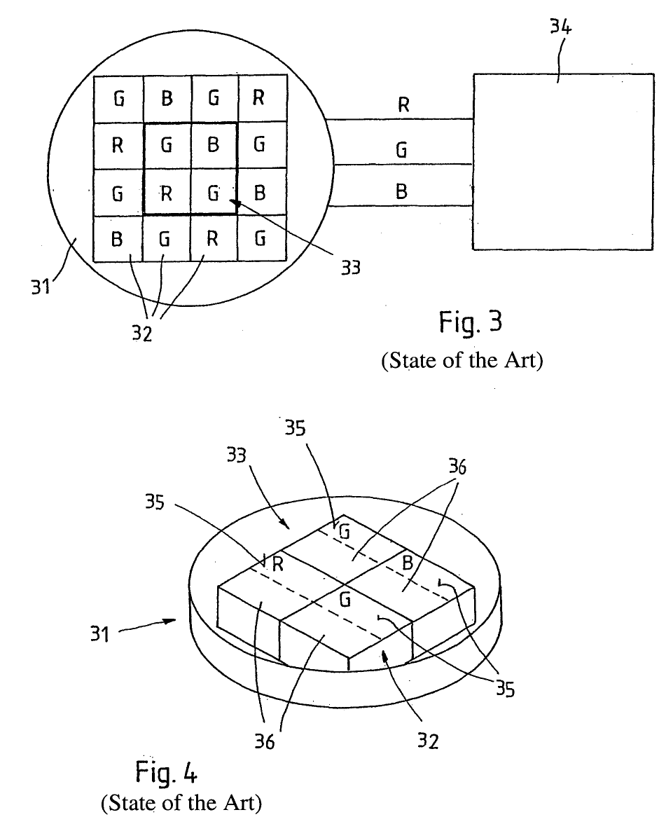

FIG. 3 Illustrates the principal structure of a CMOS used as a sensor element in accordance with the state of the art;

FIG. 4 Illustrates the sensor element in accordance with FIG. 3 in a schematic perspective view;

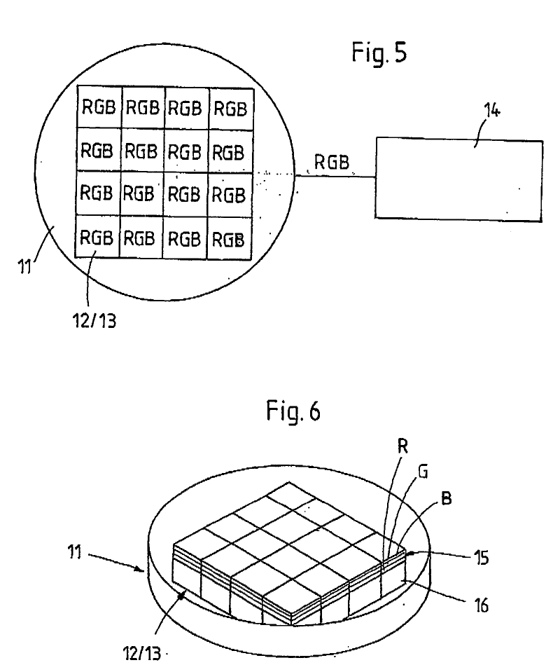

FIG. 5 Illustrates the principal structure of a sensor instrument used in accordance with the invention in endoscopic instruments;

FIG. 6 Illustrates a schematic, perspective view of a sensor element in accordance with FIG. 5;

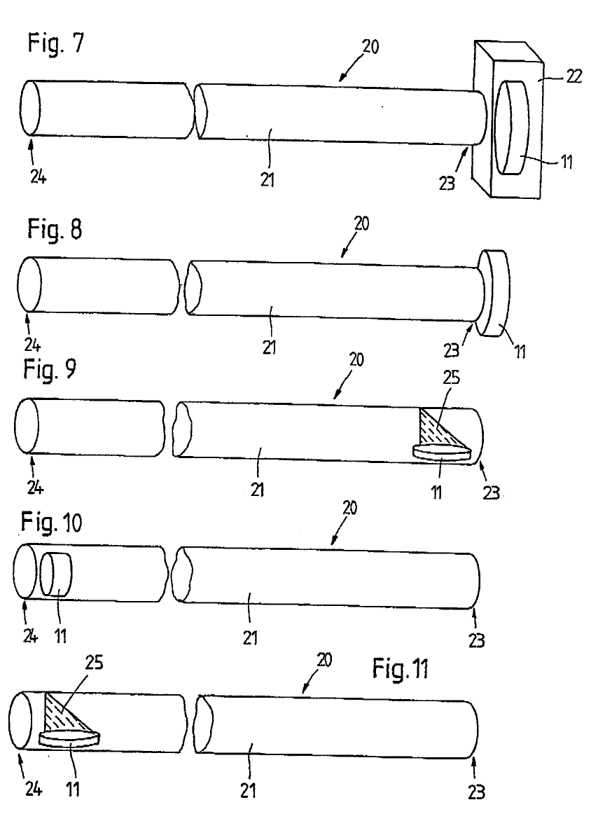

FIG. 7 Illustrates a design of an endoscopic instrument of the invention in a schematic, perspective view;

FIG. 8 Illustrates a further design of an endoscopic instrument of the invention in a schematic perspective view;

FIG. 9 Illustrates a further design of an endoscopic instrument of the invention in a schematic, perspective view;

FIG. 10 Illustrates a further design of an endoscopic instrument of the invention in a schematic, perspective view and

FIG. 11 Illustrates a further design of an endoscopic instrument of the invention in a schematic, perspective view.

A CCD (charge-coupled device) used in endoscopic instruments as a sensor element 1 in accordance with the state of the art is represented in FIGS. 1 and 2. The sensor element (CCD) 1 is comprised of several image dot units (pixels) 2 that are sensitive in various wavelength ranges, presently for the colors red R, green G and B. An image dot 3 is moreover comprised of four image dot units 2, whereby two image dot units sensitive to green light arranged diagonally in relation to each other are combined into in an image dot 3 corresponding to FIG. 1. The image dot units 2 of CCD 1 are read out cell-wise and the information from the individual image dot units 2 is fed to a processor 4 through an integrated read out control facility 2 and lines connected to the CCD 1. Since an image dot 3 assembled from four image dot units 2 is arranged in two lines of the CCD 1, but one only line can be issued serially, image information to be correspondingly displayed with image dot 3 is first possible after reading out two lines. Assembling the desired image information is consequently slow. A representation of the read out control unit 6 of sensor element 1 (CCD) is dispensed with in FIG. 2 for reasons of clarity.

A CMOS (Complementary Metal Oxide Semiconductor) used as a sensor element 31 in accordance with the state of the art is represented in FIGS. 3 and 4. The sensor element (CMOS) 31 is comprised of several image dot units (pixels), like the CCD 1, which are sensitive in various wave length ranges, presently to the colors red R, green G and blue 4. An image dot 33 is moreover comprised of four image dots 32, whereby two image dot units 32 sensitive to green light are combined in an image dot according to FIG. 3. The image dot units 32 of the CMOS 31 are read out through matrix-like addressing and the information from the individual image dot units is fed to a processor 34 for signal processing through leads connected to the CMOS 31. Owing to the division of the image dot units 32 into a detector surface 35 and a read out control unit 36, as can be recognized on the basis of FIG. 4, the sensitivity in accordance with the ratio of the read out control unit 36 diminishes toward the surface of the image dot unit 32. The surface made available to the CMOS 31 is consequently only partially usable as a detector surface for recording image information.

FIGS. 5 and 6 depict the principal structure of a sensor element 11 of an endoscopic element of the present invention. The sensor element 11 consists of an arrangement of image dot units (pixels) 12 on the basis of which the image information acting upon the sensor element 11 in the form of electromagnetic radiation can be assembled, whereby the image dot units 12 are structured axially toward the direction of incidence of the electromagnetic radiation 11 on the sensor element 11, as can be recognized on the basis of FIG. 6. Each axially-structured image dot unit, moreover, simultaneously forms an image dot 13 for recording image information. In this way, the surface furnished by the sensor element 11 is completely usable for receiving image information, owing to which, in comparison with the CCD 1 in accordance with FIGS. 1 and 2, as well as the CMOS 31 in accordance with FIGS. 3 and 4 with a constant surface of the sensor element 1, 31 or 11, basically greater resolution and sensitivity or, at identical resolution, basically smaller dimensions of the sensor element are attainable.

Through the axial structuring of the sensitive layers in the region of the sensor surface 15, at least two pieces of image information can simultaneously be detected for each image dot. Presently, three pieces of image information are detected with the sensor 15 in one image dot 13, whereby the sensor 15 has three layers sensitive to the colors red R, green G and blue B in vertical layer structure per image dot 13. The image information detected by an image dot 13 are combined by the preprocessing and reading out facility 16 and fed serially to a preprocessor 14. In this way, the number of read out lines is reduced and the computation output of the preprocessor can be conceived as a smaller design. With a refinement of the preprocessing and reading out facility 16 for increasing brightness dynamics, the quality of recordings of liquids or metals in particular can be improved that are otherwise unattainable owing to reflections. The distal ends of the sensor element otherwise constructed as frosted according to the state of the art can thus be dispensed with or the quality of the images recorded can be further improved. The brightness dynamics of ca. 60 dB existing in the state of the art when using CCDs1 as sensor elements is improvable with sensor element 12 to values of more than 120 dB.

The sensor element 11 represented in FIGS. 5 and 6 has a layer 16 with a apparatus for processing and preprocessing of image information received by the sensitive layer sequence 15 and an apparatus for emitting received processed and preprocessed image information that is fed from the apparatus for emitting contained in layer 16 to a processor 14 for further processing or preprocessing in its axial image dot units 12 structured axially in relation to the direction of incidence of the electromagnetic radiation upon the sensor element 11, thus in horizontal layer sequence. The apparatus contained in layer 16 presently encompasses an apparatus for image editing and/or evaluation, preferably in the form of lock-in amplifier facilities in addition to additional opto-electronic transducers sensitive to electromagnetic radiation. In this way, the quality of the image information detected by the image dot units 12 is improved by the elimination of noise and disturbance signals. The processor 14 connected downstream from the sensor element in series is thus relieved and can be configured in a less expensive manner, especially since the signals fed from the read out control units contained in layer 16 are qualitatively of a higher grade and the processor must only be able to preprocess more narrowband signals.

To increase sensitivity further, the surface of the sensor element 11 is constructed so that a minimization of the reflection losses on the surfaces is attained. This is attainable by variation of the layer thicknesses of the sensor elements as well as by applying one or more antireflection coatings (not represented here), for example, of magnesium fluoride or suitable, preferably dielectric multiple layer systems. Anti-reflection coatings increase sensitivity in a large wave length range, preferably in a range in which the sensor element is sensitive.

The distal probe end, in addition, includes radiation-emitting structures which are usable as an illumination facility. The processing and/or preprocessing apparatus provided for each image dot unit in layer 16, presently lock-in amplifiers, as well as the illumination facilities, permit selective signal preprocessing and editing for each image dot 13. The pixel-wise present lock-in amplifier is joined with the illumination facility as a reference signal so that foreign radiation or disturbing radiation hitting upon the sensor element have no influence upon the image information received by the sensor element 11.

Various endoscopic instruments are represented in FIGS. 7 to 11 which are constructible as flexible as well as rigid endoscopes. The image information conducting facility 21 is constructed either rigidly or flexibly for this.

With the endoscopic instrument 20 represented in FIG. 7, a sensor element 11 in accordance with FIGS. 3 and 4 is arranged in an external camera 22 which is arranged on the proximal end 23 of the endoscopic instrument 20. The image information recorded on the distal end 24 is fed through the image information conductance facility 21 to the surface of the sensor element 11 which is structured axially in relation to the direction of incidence of the electromagnetic radiation transmitting the image information.

With the endoscopic instrument in accordance with FIG. 8, the sensor element 11 is arranged at the proximal end 23 in the endoscopic instrument. The same holds for the endoscopic instrument 20 in accordance with FIG. 9, whereby the sensor element 11 is integrated here in the endoscopic instrument in the range of the proximal end 23. For this, the endoscopic instrument 20 in accordance with FIG. 9 has a radiation deflection facility 25 in the region of the proximal end 23 that feeds the image information received on the distal end 24 of the endoscopic instrument through the image conducting facility 21 to the sensor element arranged basically laterally in relation to the image information incident upon the distal end corresponding to the axial structure of the sensor element 11.

In the case of the endoscopic instrument 20 represented in FIG. 10, the sensor element 11 is arranged on the distal end 24 of the endoscopic instrument 20. The regions of the image conducting facility 21 situated in the direction of the proximal end of the endoscopic instrument 20 behind the sensor element 11 have electrical lines and the like that enable the connection of the preprocessing and/or processing processor 14 and a monitor or the like in the region of the proximal end 23.

The sensor element is arranged laterally to the image information incident in the region of the distal end in the design of an endoscopic instrument 20 represented in FIG. 11. The image information thus received in the region of the distal end 24 is deflected through a radiation deflection facility 25 and fed to the sensor element corresponding to its axial structure.

The designs represented in the Figures serve merely for the elucidation of the invention and are not restricted to this.

Reference number list

| | |
|---|---|
| 1 | Sensor element (CCD) |
| 2 | Image dot unit (pixel) |
| 3 | Image dot |
| 4 | Processor |
| 5 | Detector (image dot unit) |
| 6 | Read out control facility |
| 11 | Sensor element |
| 12 | Image dot unit (pixel) |
| 13 | Image dot |
| 14 | Processor |
| 15 | Detector (image dot unit) |
| 16 | Read out control facility/processing and/or preprocessing apparatus |
| 20 | Endoscope (rigid/flexible) |
| 21 | Image conducting facility |
| 22 | Camera |
| 23 | End (proximal) |
| 24 | End (distal) |
| 25 | Radiation deflection facility |
| 31 | Sensor element (CMOS) |
| 32 | Image point element (pixel) |
| 33 | Image dot |
| 34 | Processor |
| 35 | Detector (image dot unit) |
| 36 | Read out control facility |
| R | Red |
| G | Green |
| B | Blue |

1. Endoscopic instrument for use in hollow spaces with at least one optical system having a sensor element for receiving image information, characterized in that the sensor unit (11) consists of an arrangement of image dot units (12) from which the image information acting upon the sensor element (11) in the form of electromagnetic radiation can be assembled, whereby the image dot units (12) are structured axially in relation to the direction of incidence of the electromagnetic radiation upon the sensor element (11).

2. Endoscopic element according to claim 1, characterized in that the sensor element (11) has at least two layers (15, 16), a basically area-covering sensor layer (15) and a layer (16) serving for signal preprocessing and/or processing for each point connecting thereto in the direction of incidence of the electromagnetic radiation.

3. Endoscopic instrument according to claim 1 or claim 2, characterized in that at least two pieces of image information are detectable in an image dot unit (12) for each image dot (13).

4. Endoscopic instrument according to one of claims 1 to 3, characterized in that the image dot unit (12) is sensitive in at least two different spectral ranges.

5. Endoscopic instrument according to one of claims 1 to 4, characterized in that the sensor element (11) is constructed on an integrated circuit, especially an ASIC, preferably by at least one layer sequence sensitive toward electromagnetic radiation.

6. Endoscopic instrument according to claim 5, characterized in that the layer sequence consists of an arrangement of image dot units.

7. Endoscopic instrument according to claim 6, characterized in that each image dot unit (12) has a radiation transducer, preferably in the form of the layer sequence mentioned, for transforming incident radiation into an intensity-dependent measured value.

8. Endoscopic instrument according to claim 6 or claim 7, characterized in that each image dot unit (12) has apparatus (16) for processing and preprocessing measured values.

9. Endoscopic instrument according one of claims 6 to 8, characterized in that a read out control facility (16) is provided for a reading out of measured values related to an image dot unit in each case.

10. Endoscopic instrument according to claim 9, characterized in that reading out measured values takes place such that an image beaming onto the sensor element can be assembled from image dot unit-related measured values.

11. Endoscopic instrument according to one of the preceding claims, characterized in that each image dot unit (12) is allocated at least a further opto-electronic transducer sensitive to electromagnetic radiation.

12. Endoscopic instrument according to claim 11, characterized in that the opto-electronic transducer is a component of the integrated circuit.

13. Endoscopic instrument according to claim 11 or claim 12, characterized in that the opto-electronic transducer broadens the spectral sensitivity of the sensor in the NIR range and/or the UV range.

14. Endoscopic instrument according to one of claims 11 to 13, characterized in that the opto-electronic transducer increases the transient properties of the sensor.

15. Endoscopic instrument according to one of claims 11 to 14, characterized in that the opto-electronic transducer is a photo-diode, a photo-gate or a photo-transistor, preferably of silicon.

16. Endoscopic instrument according to one of claims 1 to 15, characterized in that the sensor element has a spectrally controllable sensitivity, preferably a multiple layer system of pilin, pipilin type or similar layer sequences.

17. Endoscopic instrument according to one of claims 1 to 16, characterized in that the integrated circuit of the sensor element (11) has an apparatus (16) for image editing and/or evaluation.

18. Endoscopic instrument according to claim 17, characterized in that the apparatus (16) for image editing and/or evaluation are devices for noise suppression, preferably by image summation and/or averaging.

19. Endoscopic instrument according to claim 18, characterized in that the apparatus (16) for image editing and/or evaluation are amplifier facilities, preferably lock-in amplifiers.

20. Endoscopic instrument according to the preceding claims, characterized in that this has at least one radiation-emitting structure which is preferably constructed as an illumination facility for lighting the hollow space or the structures to be examined.

21. Endoscopic instrument according to claim 20, characterized in that the radiation-emitting structure is/are one or more directly and/or indirectly emitting diode(s), preferably a radiation of different wavelengths.

22. Endoscopic instrument according to claim 20 or claim 21, characterized in that the radiation-emitting structure is adapted to the sensor element (11) in its spectral range and is preferably operable amplitude-modulated.

23. Endoscopic instrument according to one of the preceding claims, characterized in that the radiation-emitting structure enables a sequential irradiation of the hollow space.

24. Endoscopic instrument according to one of the preceding claims, characterized in that this is constructed as a capsular probe.

25. Endoscopic instrument according to claim 24, characterized in that the probe is independently or remotely controllable in and/or through hollow spaces, actively with a preferably integrated drive, and/or passively through peristaltic movements of organs of a body to be examined.

26. Endoscopic instrument according to claim 24 or claim 25, characterized in that the integrated circuit has a computing unit for control and/or remote control of a drive.

27. Endoscopic instrument according to claims 24 to 26, characterized in that this has a facility for non-contact transmission of received image information to an image information receiving facility situated separately outside the hollow space.

28. Endoscopic instrument according to claims 24 to 27, characterized in that the integrated circuit has a storage unit for gathering image information.

29. Endoscopic instrument according to one of claims 1 to 28, characterized in that an antireflection coating is applied on the sensor element (11), preferably of magnesium fluoride or a suitable, preferably dielectric multiple layer system.

30. Endoscopic instrument according to one of claims 1 to 29, characterized in that the sensor element (11) contains no temperature-sensitive components and is consequently autoclavable.
